(19) United States
(12) Patent Application Publication
(10) Pub. No.: US 2006/0148871 A1
(43) Pub. Date: Jul. 6, 2006

(54) Metabolic stabilization of substituted adamantane

(76) Inventors: Jeffrey J. Rohde, Evanston, IL (US); Liping Pan, Gurnee, IL (US); Marina Pliushchev, Vernon Hills, IL (US); James T. Link, Evanston, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/325,956

(22) Filed: Jan. 5, 2006

Related U.S. Application Data
(60) Provisional application No. 60/641,676, filed on Jan. 5, 2005.

(51) Int. Cl. A61K 31/41; A61K 31/19; A61K 31/18

(52) U.S. Cl. 514/381; 514/557; 514/602; 514/623

(57) ABSTRACT

The present invention is directed to the method of increasing the metabolic stability of adamantane containing compounds that are inhibitors of the 11-beta-hydroxysteroid dehydrogenase Type 1 (11-beta-HSD-1) enzyme. The stability is achieved by substitutions of the adamantane ring.

This application claims priority from U.S. Provisional Patent Application Ser. No. 60/641,676, filed Jan. 5, 2006.

### FIELD OF THE INVENTION

The present invention is directed to the method of increasing the metabolic stability of adamantane containing compounds that are inhibitors of the 11-beta-hydroxysteroid dehydrogenase Type 1 (11-beta-HSD-1) enzyme.

### BACKGROUND OF THE INVENTION

The development of new pharmaceuticals containing an adamantane ring system has been influenced by its lipophilicity that facilitates the tissue distribution of a drug containing the moiety. However, the lipophilic nature of the adamantane may also facilitate metabolic degradation, usually through oxidation. Typically, metabolic hydroxylation at any of the bridgehead carbons is the primary metabolic pathway. Replacement of the bridghead hydrogens with fluorine atoms has been claimed to increase the metabolic stability of an admantane substituted compound. Furthermore, metabolic stabilization by replacement of the bridghead hydrogens with a hydroxyl group within pharmaceutical compounds has also been reported. In some cases, these substituents are not tolerated and may not impart sufficient metabolic stabilization. The present invention describes substituents that can overcome these limitations.

### SUMMARY OF THE INVENTION

The present invention is directed to a method of increasing the metabolic stability of compounds containing an adamantane substituent that are inhibitors of the 11-beta-hydroxysteroid dehydrogenase Type 1 enzyme by substituting the adamantane as in a compound of formula (I), 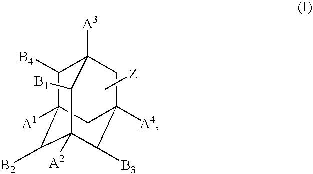
wherein

at least one of A¹, A², A³, A⁴, B¹, B², B³ and B⁴ are individually selected from the group consisting of carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl, and the remainder of A¹, A², A³, A⁴, B¹, B², B³ and B⁴ are individually selected from the group consisting of hydrogen, carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl;

R¹ and R² are each individually selected from the group consisting of hydrogen, alkyl, alkylcarbonyl, alkylsulfonyl, aryl, arylalkyl, arylcarbonyl, arylsulfonyl; and

Z is a residue which imparts 11-beta-HSD-1 activity when attached to the adamantane ring system.

In particular, adamantanes containing substituents that are charged at physiological pH, such as a carboxy substituent, exhibit increased metabolic stability. In addition, adamantanes which are substituted by other substituents that can participate in hydrogen bonding also exhibit increased metabolic stability.

To enhance the metabolic stability of a pharmaceutically active adamantane compound, in accord with the present invention, a carboxy-substituted adamantane moiety or an adamantane substituted with another substituent that will increase the stability of the adamantane containing compound, may be introduced in the pharmaceutically active adamantane compound in the place of the parent adamantane moiety.

### DETAILED DESCRIPTION OF THE INVENTION

The present invention discloses a method for increasing the metabolic stability of pharmaceutically active adamantane compound through the incorporation of an adamantane ring with at least one substituent selected from the group consisting of carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl; and R¹ and R² are each individually selected from the group consisting of hydrogen, alkyl, alkylcarbonyl, alkylsulfonyl, aryl, arylalkyl, arylcarbonyl, and arylsulfonyl.

In particular, adamantane containing groups that are charged under physiological conditions, such as but not limited to carboxy, will increase the metabolic stability of the adamantane containing compound.

[image]

The present invention contemplates the replacement of the parent adamantane with an adamantane described in FIG. 1, to increase the metabolic stability of a pharmaceutically active adamantane compound.

In addition, substituents which remain uncharged under physiological conditions, such as those shown in FIG. 2, show an increase in metabolic stability, when incorporated into adamantane containing compounds.

[image]

The present invention contemplates the replacement of the parent adamantane with an adamantane as described in FIG. 2, to increase the metabolic stability of a pharmaceutically active adamantane compound.

The present invention also contemplates increasing the metabolic stability of a pharmaceutically active adamantane compound by incorporating a substituent which can participate in hydrogen bonding.

The compounds and processes of the present invention will be better understood in connection with the following synthetic schemes and Experimentals that illustrate a means by which the compounds of the invention can be prepared.

The compounds of this invention can be prepared by a variety of procedures and synthetic routes. The substituents contemplated within the scope of this invention can be incorporated into pharmaceutical compounds using methods known to those skilled in the art Representative procedures and synthetic routes are shown in, but are not limited to, the following Schemes.

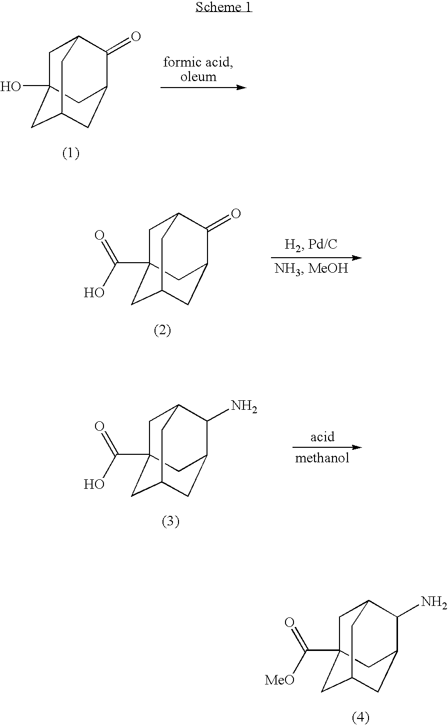

As shown in Scheme 1, alcohol (1), when treated with a mixture of formic acid and oleum, will provide acid (2). One example is the synthesis of amino ester (4) in Scheme 1. Reductive amination with ammonia in methanol over palladium on carbon under an atmosphere of hydrogen provides E- and Z-amino acid adamantane (3). Exposure of amino acid (3) to acidic methanol will provide the amino ester (4).

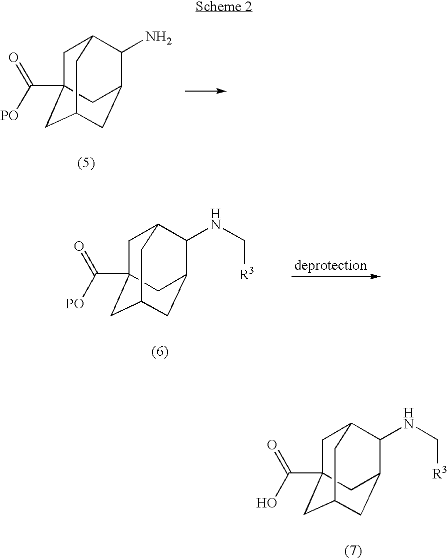

As shown in Scheme 2, amino esters (6) and related acids (7), which can be obtained from amino esters (5) using methods known to those skilled in the art, wherein P represents a protecting group, can be converted into potential pharmaceutical compounds by methods known to those in the art. For example, amino ester(5) when treated with an aldehyde of formula R³CHO [wherein R³ is a residue which imparts 11-beta-HSD-1 activity when attached to amino ester (6) and/or amino acid (7)] in the presence of a reducing agent, such as but not limited to sodium cyanoborohydride or sodium tri-acetoxyborohydride in solvents such as 1,2-dichloroethane will provide compounds of formula (6). Compound of formula (6) when deprotected using conditions known to those skilled in the art, will provide compounds of formula (7) which are representative of the compounds of the present invention.

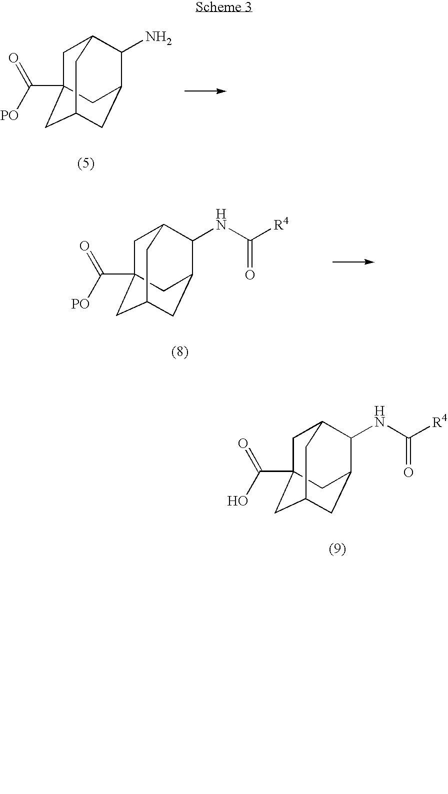

Additionally, amines of formula (5) when treated with an acid chloride of formula R⁴C(O)—Cl [wherein R⁴ is a residue which imparts 11-beta-HSD-1 activity when attached to amido ester (8) and/or amido acid (9)], in the presence of a base such as but not limited to triethylamine or N-methyl morpholine in solvents such as but not limited to dichloromethane, will provide compounds of formula (8). Alternatively, coupling of amines of formula (5) and acids of general formula R⁴C(O)—OH with reagents such as but not limited to EDCI and HOBt can provide amides of general formula (8). Similarly, compounds of formula (8) can be treated according to conditions known to deprotect esters or with methods known to those skilled in the art to provide compounds of formula (9).

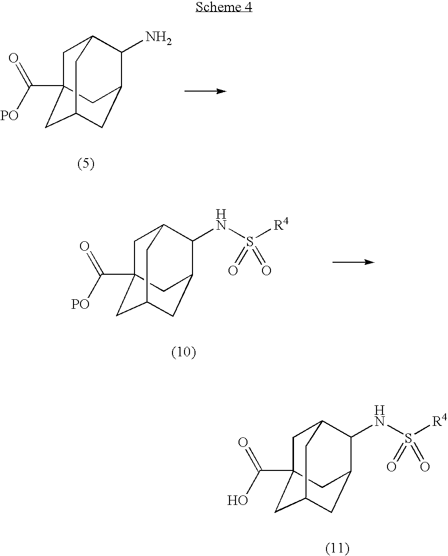

Similary, compounds of formula (5) when treated with sulfonyl chlorides according to the procedures outlined in Scheme 4 followed by conditions know to those skilled in the art to remove esters, will provide compounds of formula (11) which are representative of the compounds of the present invention.

### General Experimentals

### 4-oxo-adamantane-1-carboxylic acid

A 5L 4-neck flask equipped with N₂ inlet/bubbler with H₂O trap, overhead stirring, and an addition funnel was charged with 30% oleum (˜10.5 volumes, 2.2 L, 8×500 g bottles+100 mL), and heated to 50° C. under a slight N₂ flow. 5-Hydroxy-2-adamantanone (220 g, 81 wt % purity, 1.07 mol) was dissolved in 5 volumes HCO₂H (˜98%, 1.10 L) and added drop-wise to the warm oleum solution over 5 hours. The addition rate was adjusted to maintain the internal temperature between 70-90° C. After stirring an additional 2 hours at 70° C. The reaction solution was cooled to 10° C. in an ice bath. 20 volumes of 10% NaCl aq (4 L) were cooled to <10° C., the crude reaction mixture was quenched into the brine solution in batches, maintaining an internal temperature <70° C. The quenched reaction solution was combined with a second identical reaction mixture for isolation. The combined product solutions were extracted 3×5 volumes with CH₂Cl₂ (3×2.2 L) and the combined CH₂Cl₂ layers were then washed 1×2 volumes with 10% NaCl (1 L). The CH₂Cl₂ solution was then extracted 3x5 volumes with 10% Na₂CO₃ (3×2.2L). The combined Na₂CO₃ extracts were washed with 1×2 volumes with CH₂Cl₂ (1 L). The Na₂CO₃ layer was then adjusted to pH 1-2 with concentrated HCl (˜2 volumes, product precipitates out of solution). The acidic solution was then extracted 3×5 volumes with CH₂Cl₂ (3×2.2 L), and the organic layer was washed 1×2 volumes with 10% NaCl. The organic solution was then dried over Na₂SO₄, filtered, concentrated to ˜¼ volume, then chase distilled with 2 volumes EtOAc (1 L). Nucleation occurred during this distillation. The suspension was then chase distilled 2×5 volumes (2×2 L) with heptane and cooled to room temperature. The suspension was then filtered, and the liquors were recirculated 2× to wash the wet cake. The product was dried overnight at 50° C., 20 mm Hg to afford 397.81 g product as a white crystalline solid.

### 4-amino-adamantane-1-carboxylic acid

To 1.0 g (10 wt %) of 5% Pd/C is added 10.0 g of starting material followed by 200 mL (20 volumes) of 7M NH₃ in MeOH. The reaction mixture is stirred under an atmosphere of H₂ at RT for 16-24 hours. 200 mL of water is added and the catalyst is removed by filtration. The catalyst is washed with MeOH. Solvent is removed by distillation at a bath temperature of 35° C. until solvent stops coming over. Approximately 150 mL of a slurry remains. 300 mL of MeCN is added to the slurry, which is then stirred for three hours at RT. The slurry is filtered and washed once with 100 mL MeCN. The wet cake is dried at 50° C. and 20 mm Hg under N₂ to yield 8.65 g (86.0%) of product. The product has a 13.1:1.0 E:Z ratio5 by ¹H-NMR (D₂O).

### 4-amino-adamantane-1-carboxylic acid methyl ester

Methanol (10 volumes, 85 mL) was cooled to 0° C. AcCl was added dropwise (5.0 equiv., 15.5 mL), and the solution was warmed to ambient temperature for 15-20 minutes. E-2-amino-adamantane-5-carboxylic acid ( 8.53 g, 43.7 mmol, 1.0 equiv.) was added and the reaction solution was heated to 45° C. for 16 h (overnight). Consumption of the starting aminoacid was monitored by LC/MS (APCI). The reaction solution was then cooled to room temperature, 10 volumes MeCN (85 mL) was added, distilled to ˜¼ volume (heterogeneous), and chase distilled 2×10 volumes with MeCN (2×85 mL). The resulting suspension was cooled to room temperature, filtered, and the filtrate was recirculated twice to wash the wet cake. The product was dried at 50° C., 20 mm Hg overnight to afford the product as a white crystalline solid, 10.02 g, 93% yield.

### Experimentals

### EXAMPLE 1

### N-2-adamantyl-2-methyl-2-phenylpropanamide

A solution of 2-adamantanamine hydrochloride (38 mg, 0.20 mmol), 2-phenylisobutyric acid (30 mg, 0.19 mmol), and O-benzotriazol-1-yl-N,N,N′,N′-tetramethyluronium tetrafluoroborate (TBTU) (65 mg, 0.20 mmol) in N,N-dimethylacetamide (DMA) (2 mL) and DIEA (80 μL, 0.46 mmol) was stirred for 16 hours at 23° C. The reaction mixture was analyzed by LC/MS and determined to be near completion. The reaction mixture was concentrated under reduced pressure. The residue was dissolved in DMSO/MeOH (1:1, 1.5 mL) and purified by preparative HPLC on a Waters Symmetry C8 column (25 mm×100 mm, 7 um particle size) using a gradient of 10% to 100% acetonitrile: aqueous ammonium acetate (10 mM) over 8 min (10 min run time) at a flow rate of 40 mL/min on reverse phase HPLC to afford the title compound upon concentration under reduced pressure (11 mg, 20%). ¹H NMR (300 MHz, DMSO-d₆) δ 7.35 (m, 4H), 7.24 (m, 1H), 6.16 (d, J=6.9 Hz, 1H), 3.78 (m, 1H), 1.74 (m, 7H), 1.64 (m, 3H), 1.55 (m, 2H), 1.48 (s, 6H), 1.41 (m, 2H); MS (DCI+) m/z 298 (M+H)⁺.

### EXAMPLE 2

### N-2-adamantyl-2-(4-chlorophenyl)-2-methylpropanamide

The titled compound was prepared according to the procedure outlined in Example 1, substituting 2-(4-chloro-phenyl)-2-methyl propionic acid for 2-phenylisobutyric acid. ¹H NMR (300 MHz, DMSO-d₆) δ 7.37 (m, 4H), 6.39 (d, J=6.6 Hz, 1H), 3.78 (m, 1H), 1.76 (m, 7H), 1.66 (m, 5H), 1.47 (s, 6H), 1.42 (m, 2H); MS (DCI+) m/z 332 (M+H)⁺.

### EXAMPLE 3

### N-2-adamantyl-1-phenylcyclopropanecarboxamide

The titled compound was prepared according to the procedure outlined in Example 1, substituting 1-phenyl-cyclopropanecarboxylic acid for 2-phenylisobutyric acid. ¹H NMR (300 MHz, DMSO-d₆) δ 7.43 (m, 4H), 7.37 (m, 1H), 5.77 (d, J=7.8 Hz, 1H), 3.76 (m, 1H), 1.68 (m, 10H), 1.42 (m, 2H), 1.35 (m, 2H), 1.21 (m, 2H), 1.01 (m, 2H); MS (DCI+) m/z 296 (M+H)⁺.

### EXAMPLE 4

### N-2-adamantyl-1-(4-chlorophenyl)cyclopropanecarboxamide

The titled compound was prepared according to the procedure outlined in Example 1, substituting 1-(chloro-phenyl)-cyclopropanecarboxylic acid for 2-phenylisobutyric acid. ¹H NMR (300 MHz, DMSO-d₆) δ 7.45 (m, 4H), 5.93 (d, J=7.5 Hz, 1H), 3.77 (m, 1H), 1.69 (m, 10H), 1.46 (m, 2H), 1.34 (m, 4H), 1.01 (s, 2H); MS (DCI+) m/z 330 (M+H)⁺.

### EXAMPLE 5

### E-4-{2-Methyl-2-[4-(5-trifluoromethyl-pyridin-2-yl)-piperazin-1-yl]-propionylamino}-adamantane-1-carboxylic acid

### EXAMPLE 5A

### 2-Adamantanone-5-carboxylic acid methyl ester

A solution of 5-hydroxy-2-adamantanone (2.0 g, 12.0 mmol) in 99% formic acid (12 mL) was added dropwise with vigorous gas evolution over 40 minutes to a rapidly stirred 30% oleum solution (48 mL) heated to 60° C. (W. J. le Noble, S. Srivastava, C. K. Cheung, J. Org. Chem. 48: 1099-1101, 1983). Upon completion of addition, more 99% formic acid (12 mL) was slowly added over the next 40 minutes. The mixture was stirred another 60 minutes at 60° C. and then slowly poured into vigorously stirred methanol (100 mL) cooled to 0° C. The mixture was allowed to slowly warm to 23° C. while stirring for 2 hours and then concentrated in vacuo. The residue was poured onto ice (30 g) and methylene chloride (100 mL) added. The layers were separated, and the aqueous phase extracted twice more with methylene chloride (100 mL aliquots). The combined methylene chloride solutions were concentrated in vacuo to 50 mL, washed with brine, dried over Na₂SO₄, filtered, and concentrated in vacuo to provide the title compound as a pale yellow solid (2.5 g, 99% crude). ¹H NMR (300 MHz, DMSO-d₆) δ 3.61 (s, 3H), 2.47-2.40 (bs, 2H), 2.17-1.96 (m, 9H), 1.93-1.82 (m, 2H); MS (DCI) m/z 209 (M+H)⁺.

### EXAMPLE 5B

### E- and Z-4-Adamantamine-1-carboxylic acid methyl ester

A solution of 2-adamantanone-5-carboxylic acid methyl ester (2.0 g, 9.6 mmoles) from Example 15A and 4A molecular sieves (1.0 g) in methanolic ammonia (7N, 17 mL) was stirred overnight at room temperature. The reaction mixture was cooled in an ice bath, treated portionwise with sodium borohydride (1.46 g, 38.4 mmoles) and stirred at room temperature for 2 hours. The suspension was filtered and MeOH was removed under reduced pressure. The residue was taken into methylene chloride (200 mL) and acidified with 10% citric acid. The pH of the solution was adjusted to neutral with saturated NaHCO₃ and then saturated with NaCl. The layers were separated and the aqueous extracted twice more with methylene chloride. The combined organic extracts were dried over Na₂SO₄ and filtered. The filtrate was concentrated under reduced pressure to provide the title compound as a light yellow solid (1.7 g, 85% crude).

¹H NMR (300 MHz, CDCl₃) δ 3.66 (s, 3H), 3.16 (m, 1H), 2.27-1.46 (m, 13H); MS (DCI) m/z 210 (M+H)⁺.

### EXAMPLE 5C

### E- and Z-4-{2-Methyl-2-[4-(5-trifluoromethyl-pyridin-2-yl)-piperazin-1-yl]-propionylamino}-adamantane-1-carboxylic acid methyl ester

To a 0° C., heterogeneous solution of 2-methyl-2-[4-(5-trifluoromethyl-pyridin-2-yl)-piperazin-1-yl]-propionic acid (50 mg, 0.16 mmol) from Example 14C, E- and Z-4-adamantamine-1-carboxylic acid methyl ester (33 mg, 0.16 mmol) from Example 15B, tetrahydrofuran (1.3 mL), and Hunig's base (30 mg, 0.24 mmol) was added solid HATU (60 mg, 0.16 mmol). The stirred reaction mixture was allowed to slowly warm to 23° C. as the ice bath melted overnight (16 hours). LC/MS analysis of the homogenous reaction mixture revealed complete consumption of starting materials. The reaction mixture was concentrated under reduced pressure, and the residue purified with flash silica gel (ethyl acetate/hexanes, 20-80% gradient) to afford the title compound as a mixture of E/Z structural isomers (30 mg, 37%). Carried on as a slightly impure E/Z mixture.

### EXAMPLE 5D

### E-4-{2-Methyl-2-[4-(5-trifluoromethyl-pyridin-2-yl)-piperazin-1-yl]-propionylamino}-adamantane-1-carboxylic acid

A stirred, 23° C., homogenous solution of E- and Z-4-{2-methyl-2-[4-(5-trifluoromethyl-pyridin-2-yl)-piperazin-1-yl]-propionylamino}-adamantane-1-carboxylic acid methyl ester (19 mg, 0.037 mmol) from Example 15C and methanol (0.5 mL) became cloudy upon addition of 10% aqueous NaOH (1 mL). After stirring for 1 hour at 23° C., the reaction mixture was heated to 50° C. for 1 hour. The mixture was diluted with sat aqueous NaHCO₃ and extracted three times with a tetrahydrofuran/methylene chloride solution (4/1). The combined organic extracts were dried over Na₂SO₄, filtered, and concentrated under reduced pressure. The E/Z isomers were separated by radial chromatography with 2% methanol in ethyl acetate/hexanes (4/1) as the eluant to afford the title compound (5 mg, 27%). ¹H NMR (500 MHz, DMSO-d₆) δ 8.41 (s, 1H), 7.79 (dd, J=2.5, 9 Hz, 1H), 7.71 (d, J=7.5 Hz, 1H), 6.96 (d, J=9.5 Hz, 1H), 3.79 (m, 1H), 3.66 (m, 4H), 2.54 (m, 4H), 1.95-1.70 (m, 11H), 1.58-1.52 (m, 2H), 1.13 (s, 6H); MS (DCI) m/z 495 (M+H)⁺.

### Metabolic Stability Data

Measurement of Inhibition Constants:

The ability of test compounds to inhibit human 11β-HSD-1 enzymatic activity in vitro was evaluated in a Scintillation Proximity Assay (SPA). Tritiated-cortisone substrate, NADPH cofactor and titrated compound were incubated with truncated human 11β-HSD-1 enzyme (24-287AA) at room temperature to allow the conversion to cortisol to occur. The reaction was stopped by adding a non-specific 11β-HSD inhibitor, 18β-glycyrrhetinic acid. The tritiated cortisol that was generated was then captured by a mixture of an anti-cortisol monoclonal antibody and SPA beads coated with anti-mouse antibodies. The reaction plate was shaken at room temperature and the radioactivity bound to SPA beads was then measured on a β-scintillation counter. The 11-βHSD-1 assay was carried out in 96-well microtiter plates in a total volume of 220 μl. To start the assay, 188 μl of master mix which contains 17.5 nM ³H-cortisone, 157.5 nM cortisone, and 181 mM NADPH was added to the wells. In order to drive the reaction in the forward direction, 1 mM G-6-P was also added. Solid compound was dissolved in DMSO to make a 10 mM stock followed by a subsequent 10-fold dilution with 3% DMSO in Tris/EDTA buffer (pH 7.4). 22 μl of titrated compounds was then added in triplicate to the substrate. Reactions were initiated by the addition of 10 μl of 0.1 mg/ml E.coli lysates overexpressing 11β-HSD-1 enzyme. After shaking and incubating plates for 30 minutes at room temperature, reactions were stopped by adding 10 μl of 1 mM glycyrrhetinic acid. The product, tritiated cortisol, was captured by adding 10 μl of 1 μM monoclonal anti-cortisol antibodies and 100 μl SPA beads coated with anti-mouse antibodies. After shaking for 30 minutes, plates were read on a liquid scintillation counter Topcount. Percent inhibition was calculated based on the background and the maximal signal. Wells that contained substrate without compound or enzyme were used as the background, while the wells that contained substrate and enzyme without any compound were considered as maximal signal. Percent of inhibition of each compound was calculated relative to the maximal signal and IC₅₀ curves were generated.

Metabolic Stability Screen:

Each substrate (10 μM) was incubated with microsomal protein (0.1-0.5 mg/ml) in 50 mM potassium phosphate buffer (pH 7.4) in 48-Well plate. The enzyme reaction was initiated by the addition of 1 mM NADPH, then incubated at 37° C. in a Forma Scientific incubator (Marietta, Ohio, USA) with gentle shaking. The reactions were quenched by the addition of 800 μl of ACN/MeOH (1:1, v/v), containing 0.5 μM of internal standard (IS), after 30 min incubation. Samples were then filtered by using Captiva 96-Well Filtration (Varian, Lake Forest, Calif., USA) and analyzed by LC/MS (mass spectrometry). Liver microsomal incubations were conducted in duplicate.

In Vitro Metabolic Half-Life Study:

Example 5 (1 μM) was incubated with microsomal protein (0.5-1.0 mg/ml) in 50 mM potassium phosphate buffer (pH 7.4). After 5 minutes, preincubation at 37° C. in a shaking water bath, the enzyme reaction was initiated by the addition of 1 mM NADPH. Aliquots (200 μl) were removed and added to 100 μl of ACN/MeOH (1:1, v/v), containing 0.5 μM of IS, at the following time points: 0, 5, 10, 15, 20 and 30 min. Samples were then centrifuged at 14000×g for 10 min and the supernatant was analyzed by LC/MS. Additionally, Example 5 (1 μM) was also incubated with hepatocytes in complete culture medium (Waymouth MB 752/1). The reaction was terminated at 0, 1, 3 and 6 hours with the addition of 250 μl of ACN/MeOH (1:1, v/v). Samples were centrifuged and the supernatant was analyzed by LC/MS as described above. Liver microsomes and hepatocyte incubations were conducted in duplicate and triplicate, respectively.

LC/MS Analysis

The parent remaining in the incubation mixture was determined by LC/MS. The LC/S system consisted of an Agilent 1100 series (Agilent Technologies, Waldbronn, Germany) and API 2000 (MDS SCIEX, Ontario, Canada). A Luna C8(2) (50×2.0 mm, particle size 3 μm, Phenomenex, Torrance, Calif., USA) was used to quantify each compound at ambient temperature. The mobile phase consisted of (A): 10 mM NH₄AC (pH 3.3) and (3): 100% ACN and was delivered at a flow rate of 0.2 ml/min. Elution was achieved using a linear gradient of 0-100% B over 3 min, then held 100% B for 4 min, and returned to 100% A in 1 min. The column was equilibrated for 7 min before the next injection.

The peak area ratios (each substrate over IS) at each incubation time were expressed as the percentage of the ratios (each substrate over IS) of the control samples (0 min incubation). The parent remaining in the incubation mixture was expressed as the percentage of the values at 0 min incubation. The percentage turnover is calculated using the following equation (Y% turnover=100% turnover−X% parent remaining) and is recorded as the percentage turnover in the Table 1.

In vitro half-life of substrate depletion was determined and converted to hepatic intrinsic clearance (Obach R Scott: Prediction of human clearance of twenty-nine drugs from hepatic microsomal intrinsic clearance data: an examination of in vitro half-life approach and nonspecific binding to microsomes. Drug metabolism and Disposition. 1999, 27:1350-1359).

Microsomal Metabolism Summary

Carboxy-substituted adamantanes, and other adamantane derivatives substituted with a stabilizing substituent are more metabolically stable than adamantane containing compounds lacking those substituents. For examples, Example 1, 2, 3 and 4 are rapidly metabolized in human liver microsomes (HLM) as shown in Table 1. A compound containing a carboxy-substituted admantane (Example 5), shows excellent metabolic stability in liver microsomes at, monkey and dog (See Table 2).

TABLE 1
Liver microsomal stability

| | | HLM | MLM | RLM |
|---|---|---|---|---|
| Example | h-HSD1 IC₅₀ | % turnover | % turnover | % turnover |
| 1 | 28 | 83 | | |
| 2 | 53 | 84 | | |
| 3 | 28 | 62 | | |
| 4 | 75 | 68 | | |
| 5 | 50 | 0 | 0 | 0 |

HLM is human liver microsomes,
MLM is mouse liver microsomes, and
RLM is rat liver microsomes.

In addition to robust metabolic stability in human, mouse, rat, monkey, and dog microsomes, hepatocyte stability across five species is also high.

TABLE 2
Liver microsomal and hepatocyte intrinsic clearance
(CLint in L/hr · kg) data for Example 5.

| Assay | human | mouse | rat | monkey | dog |
|---|---|---|---|---|---|
| Microsomes | .29 | 7.61 | 2.4 | 1.89 | .45 |
| Hepatocytes | .09 | .82 | .31 | .28 | .22 |

CLint in L/hr · kg

The metabolic stability data of Table 1 and Table 2, demonstrates that an adamantane compound of formula (I) contains substituents that impart an increase in metabolic stability compared to an adamantane containing compound which lack those substituent. This increase in metabolic stability may lead to longer in vivo halflive and a pharmacokinetic advantage.

Compounds of formula (I), which contain substituents which imparts metabolic stability, are stable 11-β-hydroxysteroid dehydrogenase type 1 inhibitors. The Compounds of formula (I) may be used for the treatment or prevention of non-insulin dependent type 2 diabetes, obesity, dyslipidemia insulin resistance, metabolic syndrome, and/or any condition exacerbated or caused by glucocorticoid excess.

What is claimed:

1. A method of increasing the metabolic stability of a pharmaceutically active adamantane compound by incorporating a substituted adamantane ring of formula (I), 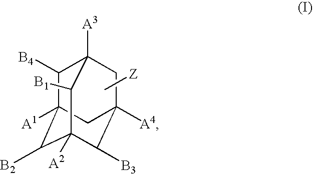
wherein
one or more of A¹, A², A³, A⁴, B¹, B², B³ and B⁴ are individually selected from the group consisting of carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl, and the remainder of A¹, A², A³, A⁴, B¹, B², B³ and B⁴ are individually selected from the group consisting of hydrogen, carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl;
R¹ and R² are each individually selected from the group consisting of hydrogen, alkyl, alkylcarbonyl, alkylsulfonyl, aryl, arylalkyl, arylcarbonyl, arylsulfonyl; and
Z is a residue which imparts 11-beta-HSD-1 activity when attached to the adamantane ring system.

2. A method according to claim 1, wherein at least one of A¹, A², A³, A⁴, B¹, B², B³ and B⁴ are individually selected from the group consisting of carboxy, alkyl-S(O)₂NHC(O)—, tetrazolyl, carboxyalkyl and R¹R²N-alkyl, and the remainder of A¹, A², A³, A⁴, B¹, B², B³ and B⁴ are individually selected from the group consisting of hydrogen, carboxy, alkyl-S(O)₂NHC(O)—, tetrazolyl, carboxyalkyl and R¹R²N-alkyl;
R¹ and R² are each individually selected from the group consisting of hydrogen, alkyl, alkylcarbonyl, alkylsulfonyl, aryl, arylalkyl, arylcarbonyl, arylsulfonyl; and
Z is a residue which imparts 11-beta-HSD-1 activity when attached to the adamantane ring system.

3. A method according to claim 1, wherein at least one of A¹, A², A³, A⁴, B¹, B², B³ and B⁴ are individually selected from the group consisting of R¹C(O)—N(R²)—, R¹S(O)₂N(R²)—, R¹R²N-alkyl, R¹R²NC(O)—, and R¹R²NC(O)-alkyl, and the remainder of A¹, A², A³, A⁴, B¹, B², B³ and B⁴ are individually selected from the group consisting of hydrogen, R¹C(O)—N(R²)—, R¹S(O)₂N(R²)—, R¹R²N-alkyl, R¹R²NC(O)—, and R¹R²NC(O)-alkyl;
R¹ and R² are each individually selected from the group consisting of hydrogen, alkyl, alkylcarbonyl, alkylsulfonyl, aryl, arylalkyl, arylcarbonyl, arylsulfonyl; and
Z is a residue which imparts 11-beta-HSD-1 activity when attached to the adamantane ring system.

4. A method of increasing the metabolic stability of a pharmaceutically active adamantane compound that inhibits 11-beta-hydroxysteroid dehydrogenase Type 1 (11-beta-HSD-1) enzyme by incorporating a substituted adamantane ring of formula (I), 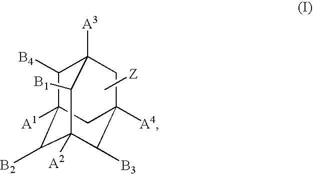
wherein
one or more of A¹, A², A³, A⁴, B¹, B², B³ and B⁴ are individually selected from the group consisting of carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl, and the remainder of A¹, A², A³, A⁴, B¹, B², B³ and B⁴ are individually selected from the group consisting of hydrogen, carboxy, alkyl-S(O)₂NHC(O)—, tetrazolyl, carboxyalkyl, R¹C(O)—N(R²)—, R¹S(O)₂N(R²)—, R¹R²N-alkyl, R¹R²NC(O)—, and R¹R²NC(O)-alkyl;
R¹ and R² are each individually selected from the group consisting of hydrogen, alkyl, alkylcarbonyl, alkylsulfonyl, aryl, arylalkyl, arylcarbonyl, and arylsulfonyl; and
Z is a residue which imparts 11-beta-HSD-1 activity when attached to the adamantane ring system.

5. A method of increasing metabolic stability of a pharmaceutically active adamantane compound by substituting the adamantane compound with a group that can participate in hydrogen bonding.
